(19) United States
(12) Patent Application Publication
(10) Pub. No.: US 2002/0115971 A1
(43) Pub. Date: Aug. 22, 2002

(54) Thin, high capacity multi-layer absorbent core

(76) Inventors: Lori Tassone Holmes, Neenah, WI (US); Michael John Niemeyer, Appleton, WI (US); Lawrence Howell Sawyer, Neenah, WI (US); Robert Alan Stevens, Appleton, WI (US)

Correspondence Address: Pauley Petersen Kinne & Fejer, Suite 365, 2800 W. Higgins Road, Hoffman Estates, IL, 60195, US

(21) Appl. No.: 09/939,535

(22) Filed: Aug. 24, 2001

Related U.S. Application Data
(60) Provisional application No. 60/257,328, filed on Dec. 20, 2000.

(51) Int. Cl. A61F 13/15; A61F 13/20

(52) U.S. Cl. 604/378; 604/379; 604/375

(57) ABSTRACT

A thin, high capacity, multi-layer absorbent material. An upper layer displays intake properties while a lower layer displays distribution properties. The upper and lower layers are both high density combinations of fluff pulp and superabsorbent material, with the lower layer having a higher density than the upper layer. The upper layer is suitably drum-formed, the lower layer is suitably air-laid, and additional layers can be either inserted between the upper and lower layers or added on top of the upper layer. The absorbent material can be used to provide discreet absorbent protection in absorbent articles, such as diapers, training pants, feminine hygiene products, incontinence products, and swim wear.

### FIELD OF THE INVENTION

This invention is directed to a thin, high density, high capacity, multi-layer absorbent material, and a method of making such material.

### BACKGROUND OF THE INVENTION

Many known absorbent composites that include a high-absorbency material include the high-absorbency material in relatively low concentrations. That is, many of the absorbent composites include airlaid cellulosic fibers and less than about 20 weight percent of a high-absorbency material. This is due to several factors.

Many high-absorbency materials are unable to absorb a liquid at a rate at which the liquid is applied to the absorbent composites during use. Accordingly, a relatively high concentration of fibrous material is desirable to temporarily hold the liquid until the high-absorbency material can absorb it. Further, the fibers serve to separate the particles of high-absorbency material so that gel-blocking does not occur. Gel-blocking refers to the situation wherein particles of high-absorbency material deform during swelling and block the interstitial spaces between the particles, or between the particles and the fibers, thus preventing the flow of liquid through the interstitial spaces.

U.S. Pat. No. 5,147,343 issued Sep. 15, 1992, to Kellenberger describes an absorbent composite adapted to avoid the problem of gel-blocking. U.S. Pat. No. 5,147,343 describes the use of a superabsorbent material which can absorb at least 27 milliliters of a 0.9 weight percent aqueous sodium chloride solution per gram of superabsorbent material while the superabsorbent is under a restraining pressure of at least 21,000 dynes per square centimeter. When the superabsorbent material is in the form of discrete particles, at least about 50 percent by weight of the superabsorbent material has a size greater than the median pore size of the porous fiber matrix when wet. The described absorbent composites are said to contain up to about 90 weight percent of a superabsorbent material.

The presence of a relatively low concentration of high-absorbency material and a relatively greater concentration of fibrous materials has resulted in the production of absorbent composites which tend to be relatively thick. In some instances, the use of a relatively thick absorbent composite in a disposable absorbent garment is acceptable. However, in recent years it has become increasingly desirable to produce absorbent composites which are thin compared to the more traditional ;absorbent composites but which still possess the same absorbent capacity. In particular, discretion and leakage are top concerns from parents and users of youth and adult incontinence products. Furthermore, unlike infant and toddler products, youth and adult pants require maximum capacity in a target zone for heavy wetting. Therefore, absorbent composites that are thin and have a high intake ability are highly desirable.

The desire to produce relatively thin absorbent composites has resulted in the desire to incorporate ever-increasing amounts of high-absorbency material into the absorbent composites. This is because the absorbent capacity of such high-absorbency materials is generally many times greater than the absorbent capacity of fibrous materials. For example, a fibrous matrix of wood pulp fluff can absorb about 7-9 grams of a liquid, (such as 0.9 weight percent saline) per gram of wood pulp fluff, while the high-absorbency materials can absorb at least about 15, preferably at least about 20, and often at least about 25 grams of liquid, such as 0.9 weight percent saline, per gram of the high-absorbency material.

U.S. Pat. No. 5,149,335 issued Sep. 22, 1992, to Kellenberger et al. is directed to an absorbent structure containing a relatively high concentration of superabsorbent material. Specifically, U.S. Pat. No. 5,149,335 describes the use of a superabsorbent material having certain absorbent characteristics when it is desired to employ the superabsorbent material at relatively high concentrations. Specifically, the superabsorbent material is described as having a 5-minute Absorbency Under Load value of at least about 15 grams per gram and a free-swell rate of less than about 60 seconds.

U.S. Pat. No. 5,866,242 issued Feb. 2, 1999, to Tan et al. is directed to a multi-layer absorbent material in which at least one layer is free of superabsorbent material.

In striving for thin absorbent composites, other desirable qualities are often sacrificed, such as fluid intake performance, capacity and flexibility. Quite often, when absorbent material is densified to create high capacity in a thin form, hard spots develop within the material, thereby resulting in stiffness within the material. On the other hand, when thin materials are made having a lower density, the resulting materials may be flexible, but thin, low density materials have a low absorbent capacity. Low density, high capacity materials that are flexible are generally thick and bulky and look and feel cumbersome on the wearer.

There is thus a need or desire for an absorbent material that is thin and has a high fluid absorption capacity.

There is a further need or desire for an absorbent garment having a discreet absorbent layer with maximum fluid absorption capacity in a target zone.

### SUMMARY OF THE INVENTION

The present invention is directed to an absorbent material made of multiple high density layers of flexible material having a high superabsorbent concentration to achieve a notably thin product with high fluid absorption capacity, and a method of making such absorbent materials. An upper layer maintains intake properties and is designed to have a lower density than a distributing lower retention layer. The upper layer is a drum-formed composite of pulp fluff and superabsorbent material (SAM). The lower layer is an air-laid composite of pulp fluff and SAM that is cut and placed in desired locations of the multi-layer absorbent material. The combination of the high density air-laid lower layer combined with the drum-formed upper layer allows a soft and flexible material to achieve conformability and comfort while reducing bulk and maintaining intake capability with full capacity. Additional absorbent layers, similar to either the upper layer or the lower layer can be inserted between the upper and lower layers. Once the multi-layer absorbent material is formed, the composite absorbent material can be wrapped in tissue and densified.

The absorbent material has a bulk thickness between about 1 and 7 millimeters, and an absorbent capacity, measured using the liquid saturation capacity test procedure described herein, between about 14 and 40 grams of 0.9 weight percent sodium chloride solution per gram of absorbent material. Suitably, the absorbent material has an absorbent capacity of at least 16, more suitably at least 18 grams of 0.9 weight percent sodium chloride solution per gram of absorbent material. The upper layer can contain about 20 to 80 weight percent of superabsorbent material and can have a density range of 0.1 to 0.4 grams per cubic centimeter. The lower layer can contain about 10 to 80 weight percent of superabsorbent material and can have a density range of 0.2 to 0.5 grams per cubic centimeter. The upper layer, having a high concentration of superabsorbent material and high density, retains good intake performance and works synergistically with the very high density lower layer.

The absorbent material can be made by drum-forming the upper layer and bonding the drum-formed upper layer to an offline-formed, such as an airlaid, lower layer.

With the foregoing in mind, it is a feature and advantage of the invention to provide a discreetly thin, multi-layer, high capacity absorbent material. It is another feature and advantage of the invention to provide a discreetly thin, multi-layer, high capacity absorbent material that can provide maximum absorbent capacity in target zones of absorbent garments.

### BRIEF DESCRIPTION OF THE DRAWINGS

FIG. 1 is a perspective view of one embodiment of an absorbent material of the invention;

FIG. 2 is a perspective view of another embodiment of an absorbent material of the invention;

FIG. 3 is a perspective view of yet another embodiment of an absorbent material of the invention;

FIG. 4 is a plan view of a child's training pant in a partially disassembled, stretched flat state, showing the surface of the article that faces the wearer when the article is worn, and with portions cut away to show the underlying features including an absorbent material;

FIG. 5 is a plan view of apparatus used to make an absorbent material;

FIG. 6 is a top view of an absorbent pad test sample used in Example Set 1;

FIG. 7 is a top view of an absorbent pad control sample used in Example Set 1;

FIG. 8 is an illustration of equipment for determining the liquid saturated retention capacity of an absorbent structure;

FIG. 9 is an illustration of equipment for determining the Absorbency Under Load (AUL) of superabsorbent material;

FIGS. 10 and 11 are illustrations of equipment for determining the Superabsorbent Gel Bed Permeability (GBP); and

FIG. 12 is an illustration of equipment for determining intake and runoff properties of an absorbent structure.

### DEFINITIONS

Within the context of this specification, each term or phrase below will include the following meaning or meanings.

“Air-laid” refers to a process for making material wherein fibers, such as cellulose-type fibers, and superabsorbent materials are arranged on a wire as a base sheet, and where the base sheet can be sprayed with an adhesive or combined with heat-activated fibers, powders, or the like. Alternatively, the base sheet can be calendered with sufficient heat and pressure to produce significant hydrogen bonding between base sheet components. The air-laid material is thus a bonded material.

“Base sheet” refers to an absorbent material web from an off-line material production process for forming wide width absorbent materials. The base sheet width is generally multiples of the absorbent material width used in an absorbent product. The base sheet is usually slit to widths appropriate for absorbent products and rewound into a roll good or package appropriate for the converting process.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Drum-formed” refers to an online process that is an integral part of a consumer product converting operation for making materials wherein fibers, such as cellulose-type fibers, and superabsorbent particles, are formed into a cohesive layer within a drum former.

“High gel strength” refers to a material having a gel strength value greater than 0.65, suitably greater than 0.75, or suitably greater than 0.85, wherein gel strength is determined by dividing 0.9 AUL capacity by centrifuge retention capacity (CRC).

“Layer,” when referring to a single layer, refers to a single planar element having a thickness devoid of any interfaces between the single planar element and any other planar elements within the thickness.

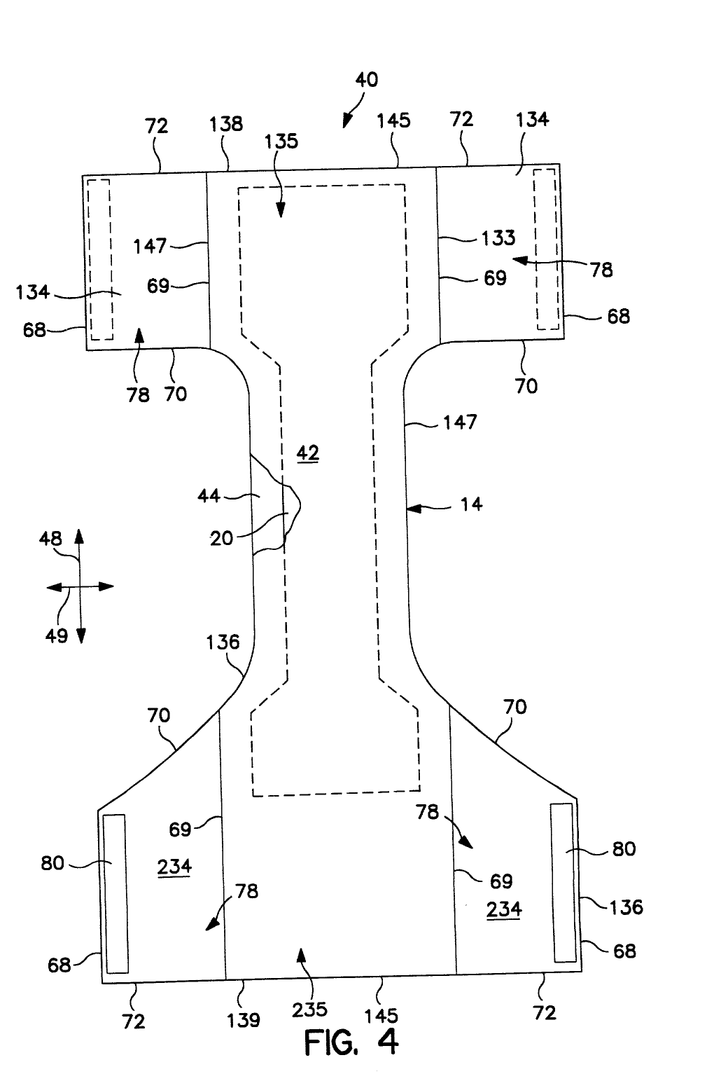

“Longitudinal” and “transverse” have their customary meaning, as indicated by the longitudinal and transverse axes depicted in FIG. 4. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Lower layer” refers to a layer of absorbent material that tends to absorb and distribute liquid within a plane of the layer and retain the liquid therein. The lower layer is typically located below an upper layer, with the upper layer in closer proximity to incoming liquid. The lower layer can have a higher density than the upper layer.

“Meltblown fibers” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self-bonding when deposited onto a collecting surface. Meltblown fibers used in the present invention are preferably substantially continuous in length.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Pulp fluff” or “fluff pulp” refers to a material made up of cellulose fibers. The fibers can be either natural or synthetic, or a combination thereof. The material is typically lightweight and has absorbent properties.

“Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

“Upper layer” refers to a layer of absorbent material that tends to intake liquid, to retain a portion of the liquid, and to allow a lower layer to desorb the remaining liquid, where the lower layer is located beneath the upper layer and opposite the direction of incoming liquid. The upper layer can have a lower density than the lower layer.

These terms may be defined with additional language in the remaining portions of the specification.

### DETAILED DESCRIPTION OF THE PRESENTLY PREFERRED EMBODIMENTS

The present invention is directed to an absorbent material made of multiple high density layers of flexible material having a high superabsorbent concentration. The absorbent material of the present invention can suitably be incorporated into absorbent articles. The term “absorbent article” includes without limitation diapers, training pants, swim wear, absorbent underpants, baby wipes, incontinence products, feminine hygiene products and medical absorbent products (for example, absorbent medical garments, underpads, bandages, drapes, and medical wipes). As used herein, the term “incontinence products” includes absorbent underwear for children, absorbent garments for children or young adults with special needs such as autistic children or others with bladder/bowel control problems as a result of physical disabilities, as well as absorbent garments for incontinent older adults.

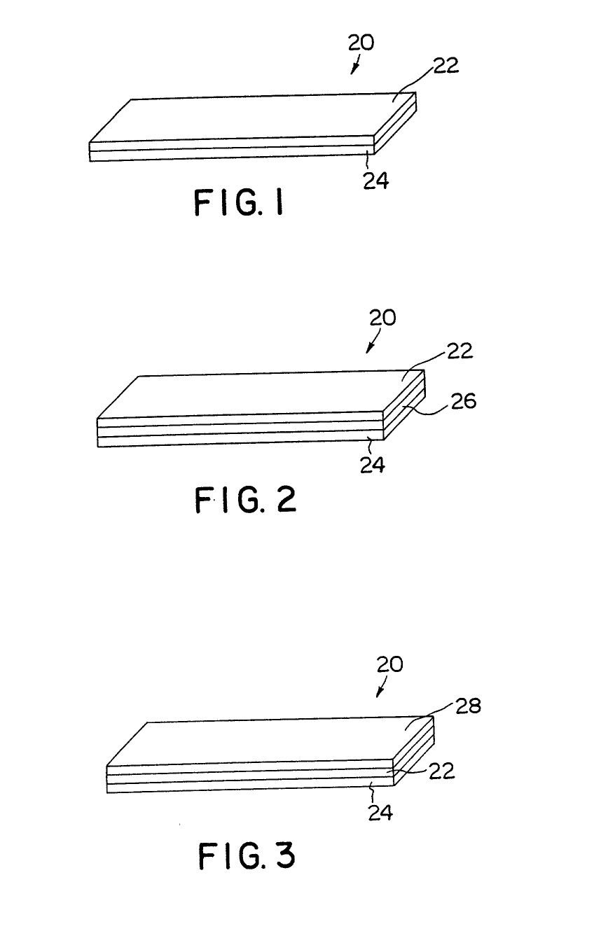

Referring to FIG. 1, a sample of absorbent material 20 of the present invention is illustrated. The absorbent material 20 includes an upper layer 22 which is configured to face and/or contact a wearer, and a lower layer 24 adjacent the upper layer 22. In an alternative embodiment shown in FIG. 2, an intermediate layer 26 can be located between the upper layer 22 and the lower layer 24. In yet another embodiment shown in FIG. 3, an additional layer 28 can be located on top of the upper layer 22. In addition to the upper layer 22 and the lower layer 24, the absorbent material 20 can include virtually any number of intermediate layers 26 and/or additional layers 28, as long as the absorbent material 20 does not become too bulky. The upper layer 22 is generally designed to maintain intake properties, while the lower layer 24 is generally designed to maintain distribution and retention properties. Thus, the lower layer 24 is normally designed to have a higher density than the upper layer 22 since fluid intake performance tends to decrease at higher density levels. However, the upper layer may have an equivalent or higher density than the lower layer as long as overall product performance is maintained. The upper layer 22 suitably has a high concentration of superabsorbent material and high density yet retains good intake performance and works synergistically with the high density lower layer 24.

In yet another embodiment of the invention, the lower layer 24 can be smaller than the upper layer 22 and may be discontinuous. That is, the lower layer 24 can be cut into a smaller, specifically shaped piece or cut into several pieces and placed in areas most in need of high absorption capacity, thereby minimizing bulk thickness in areas in which high absorption capacity is not needed.

The absorbent material 20 is generally compressible, conformable, non-irritating to a wearer's skin, and capable of absorbing and retaining liquids and certain body wastes. Each layer of the absorbent material 20 contains high levels of superabsorbent material (SAM), mixed with cellulose fluff pulp. In the upper layer 22, superabsorbent levels can range from about 20 to about 80 weight percent of the layer 22, suitably between 25 and 75 wt %, more suitably between 30 and 70 wt % based on total weight of the layer 22. Consequently, levels of fluff pulp can range between 20 and 80 wt %, suitably between 25 and 75 wt %, more suitably between 30 and 70 wt % based on total weight of the layer 22. In the lower layer 24, superabsorbent levels can range from about 10 to about 80 weight percent of the layer 24, suitably between 15 and 80 wt %, more suitably between 20 and 80 wt % based on total weight of the layer 24. Consequently, levels of fluff pulp can range between 20 and 90 wt %, suitably between 20 and 85 wt %, more suitably between 20 and 80 wt % based on total weight of the layer 24. Any intermediate layer 26 or additional layer 28 suitably has a concentration range of superabsorbent material comparable to the levels found in either the upper layer 22 or the lower layer 24.

The absorbent material 20 has a bulk thickness of between about 0.5 and 7.5 millimeters (mm), suitably between about 1 and 7 mm. The bulk thickness of the upper layer 22 is suitably between about 0.2 and 6.8 mm, more suitably between about 0.3 and 6.5 mm. The bulk thickness of the lower layer 24 is suitably between about 0.3 and 6.8 mm, more suitably between about 0.5 and 6.7 mm.

As mentioned, the density of the upper layer 22 may be less than the density of the lower layer 24 to allow the upper layer 22 to provide maximum intake. Suitably, the difference in density between the upper and lower layers is about 0 to 0.4 grams per cubic centimeter (g/cc), more suitably about 0.05 to 0.35 g/cc, desirably about 0.15 to 0.25 g/cc. More particularly, the density of the upper layer 22 is in a range of about 0.05 to 0.45 g/cc, suitably between about 0.1 and 0.4 g/cc. The density of the lower layer 24 is in a range of about 0.15 to 0.55 g/cc, suitably between about 0.2 and 0.5 g/cc. Any intermediate layer 26 or additional layer 28 suitably has a density comparable to the density of either the upper layer 22 or the lower layer 24. For example, in one embodiment, the additional layer 28 can have the same or similar density as the lower layer 24, with the upper layer 22 between the additional layer 28 and the lower layer 24 such that the additional layer 28 and the lower layer 24 both act as distribution layers while the upper layer 22 acts as an intake layer.

In other cases, the upper layer 22 may have a higher density than the lower layer. The density difference may be 0.0 grams per cubic centimeter (g/cc) to about 0.2 g/cc.

The superabsorbent material used in the absorbent material 20 of the present invention must be able to absorb a liquid under an applied load. As used herein, the Absorbency Under Load (AUL) value of a particular superabsorbent material refers to the amount, in grams, of an aqueous solution of sodium chloride (0.9 weight percent sodium chloride) which 1 gram of superabsorbent material can absorb in 60 minutes while under a given restraining load.

The absorbent material 20 of the invention suitably has an absorbent saturation capacity between about 14 and 40 grams fluid/gram absorbent material, more suitably between about 16 and 38 grams/gram, most suitably between about 18 and 32 grams/gram. More particularly, the upper layer 22 suitably has an absorbent capacity between about 14 and 40 grams/gram, more suitably between about 16 and 38 grams/gram, most suitably between about 18 and 32 grams/gram. The lower layer 24 suitably has an absorbent capacity between about 14 and 40 grams/gram, more suitably between about 16 and 32 grams/gram, most suitably between about 18 and 32 grams/gram. The method by which the absorbent saturation capacity is determined is set forth in detail below.

The cellulose fluff pulp suitably includes wood pulp fluff. The wood pulp fluff can be exchanged with synthetic, polymeric, meltblown fibers or with a combination of meltblown fibers and natural fibers. One preferred type of fluff is identified with the trade designation CR1654, available from U.S. Alliance, Childersburg, Ala., U.S.A., and is a bleached, highly absorbent sulfate wood pulp containing primarily soft wood fibers. A special densification pulp, identified with the trade designation ND-416, available from Weyerhaeuser of Federal Way, Wash., U.S.A., may offer some process benefits.

Suitable superabsorbent materials can be selected from natural, synthetic, and modified natural polymers and materials. The superabsorbent materials can be inorganic materials, such as silica gels, or organic compounds, including natural materials such as agar, pectin, guar gum, and the like, as well as synthetic materials, such as synthetic hydrogel polymers. Such hydrogel polymers include, for example, alkali metal salts of polyacrylic acids; polyacrylamides; polyvinyl alcohol; ethylene maleic anhydride copolymers; polyvinyl ethers; hydroxypropylcellulose; polyvinyl morpholinone; polymers and copolymers of vinyl sulfonic acid, polyacrylates, polyacrylamides, polyvinyl pyridine; and the like. Other suitable polymers include hydrolyzed acrylonitrile grafted starch, acrylic acid grafted starch, and isobutylene maleic anhydride copolymers and mixtures thereof. The hydrogel polymers are preferably lightly crosslinked to render the material substantially water-insoluble. Crosslinking may, for example, be by irradiation or by covalent, ionic, Van der Waals, or hydrogen bonding. The superabsorbent materials may be in any form suitable for use in absorbent structures, including particles, fibers, flakes, spheres, and the like. In one embodiment of the present invention, the superabsorbent material includes particles of a hydrocolloid, preferably an ionic hydrocolloid.

Typically, a superabsorbent material is capable of absorbing at least about 15 times its weight in water, and desirably is capable of absorbing more than about 25 times its weight in water. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany. One particular SAM useful in this invention is FAVOR® SXM 9543, available from Stockhausen GmbH & Co. This SAM provides both good processability and functional performance in the absorbent pad 20 of the invention. Other suitable types of superabsorbent materials are described in U.S. Pat. No. 5,601,542 issued Feb. 11, 1997, to Melius et al.; U.S. patent application Ser. No. 09/475,829 filed in Dec. 1999 and assigned to Kimberly-Clark Corporation; and U.S. patent application Ser. No. 09/475,830 filed in Dec. 1999 and assigned to Kimberly-Clark Corporation; each of which is hereby incorporated by reference.

Exemplary of specific superabsorbent materials suitable for use in the present invention are polyacrylate materials obtained from Stockhausen under the designations FAVOR® SXM 77 and FAVOR® SXM 880, as well as polyacrylate materials obtained from Dow Chemical, USA under the designation of DryTech 2035. Gel strength and permeability data for these superabsorbent materials are listed in Table 1.

TABLE 1
Superabsorbent Gel Strength and Permeability

| Superabsorbent | Gel Strength | Permeability (e⁻⁹cm²) |
|---|---|---|
| FAVOR ® SXM 9543 | 0.9 | 300 |
| FAVOR ® SXM 880 | 0.7 | 80 |
| FAVOR ® SXM 77 | 0.6 | 15 |
| DryTech 2035 | 0.4 | 40 |

The superabsorbent material can be in the form of particles which, in the unswollen state, have maximum cross-sectional diameters within the range of from about 50 microns to about 1,000 microns, preferably within the range of from about 100 microns to about 800 microns, as determined by sieve analysis according to American Society for Testing Materials (ASTM) Test Method D-1921. It is understood that the particles of superabsorbent material, falling within the ranges described above, may include solid particles, porous particles, or may be agglomerated particles including many smaller particles agglomerated into particles within the described size ranges. The absorbent material 20 can suitably be wrapped or encompassed by a tissue wrap that maintains the integrity and/or shape of the absorbent material.

FIG. 4 shows a child's training pant 40 in a partially disassembled, stretched flat state with the absorbent material of the invention incorporated therein, showing a surface of the training pant that faces the wearer when the garment is worn. An absorbent chassis 14 defines a pair of transversely opposed side edges 136 and a pair of longitudinally opposed waist edges, which are designated front waist edge 138 and back waist edge 139. When the training pant is in a fastened position (not shown), the absorbent chassis also defines a waist opening along the front waist edge 138 and the back waist edge 139 and two leg openings along the transversely opposed side edges 136. The chassis 14 also includes a somewhat rectangular composite structure 133, a pair of transversely opposed front side panels 134, and a pair of transversely opposed back side panels 234. The composite structure 133 and side panels 134 and 234 may be integrally formed, or may include two or more separate elements, as shown in FIG. 4.

The illustrated composite structure 133 includes an outer cover 44, a body side liner 42 which is connected to the outer cover in a superposed relation, and the absorbent material 20 of the invention which is located between the outer cover 44 and the body side liner 42. The rectangular composite structure 133 has opposite linear end edges 145 that form portions of the front and back waist edges 138 and 139, and opposite linear, or curvilinear, side edges 147 that form portions of the side edges 136 of the absorbent chassis 14. For reference, arrows 48 and 49 depicting the orientation of the longitudinal axis and the transverse axis, respectively, of the training pant 20 are illustrated in FIG. 4.

The liquid permeable body side liner 42 is illustrated as overlying the outer cover 44 and the absorbent material 20 (FIG. 4), and may but need not have the same dimensions as the outer cover 44. The body side liner 42 is desirably compliant, soft feeling, and non-irritating to the child's skin. Further, the body side liner 42 can be less hydrophilic than the absorbent material 20, to present a relatively dry surface to the wearer and permit liquid to readily penetrate through its thickness. The absorbent material 20 (FIG. 4) is positioned between the outer cover 44 and the body side liner 42, which components can be joined together by any suitable means, such as adhesives, as is well known in the art.

The absorbent chassis 14 can also incorporate other materials that are designed primarily to receive, temporarily store, and/or transport liquid along the mutually facing surface with the absorbent material 20, thereby maximizing the absorbent capacity of the absorbent material 20. One suitable material is referred to as a surge layer (not shown) and can be, for example, a material having a basis weight of about 50 grams per square meter, and can include a through-air-bonded-carded web of a homogenous blend of 60 percent 3 denier bicomponent fiber including a polyester core/polyethylene sheath, commercially available from KoSa Corporation, and 40 percent 6 denier polyester fiber, commercially available from KoSa Corporation, in Salisbury, N.C., U.S.A.

The outer cover 44 desirably includes a material that is substantially liquid impermeable, and can be elastic, stretchable or nonstretchable. The outer cover 44 can be a single layer of liquid impermeable material, but desirably includes a multi-layered laminate structure in which at least one of the layers is liquid impermeable. For instance, the outer cover 44 can include a liquid permeable outer layer and a liquid impermeable inner layer that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. The outer layer may also be made of those materials of which liquid permeable bodyside liner 42 is made. While it is not a necessity for the outer layer to be liquid permeable, it is desired that it provides a relatively cloth-like texture to the wearer.

The inner layer of the outer cover 44 can be both liquid and vapor impermeable, or can be liquid impermeable and vapor permeable. The inner layer is desirably manufactured from a thin plastic film, although other flexible liquid impermeable materials may also be used. The inner layer, or the liquid impermeable outer cover 44 when a single layer, prevents waste material from wetting articles, such as bedsheets and clothing, as well as the wearer and caregiver. A suitable liquid impermeable film for use as a liquid impermeable inner layer, or a single layer liquid impermeable outer cover 44, is a 0.02 millimeter polyethylene film commercially available from Huntsman Packaging of Newport News, Va., U.S.A. If the outer cover 44 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 44. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn. Other similar materials with varying degrees of liquid permeability are spunbond meltblown webs, spunbond/meltblown/spunbond hydrophobic, uniformly formed spunbond, or bi-component webs. A balance of barrier and permeability can be adjusted with fiber size and basis weight.

The bodyside liner 42 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 42. For example, the bodyside liner can be composed of a meltblown or spunbonded web of polyolefin fibers. The bodyside liner can also be a bonded-carded web composed of natural and/or synthetic fibers. The bodyside liner can be composed of a substantially hydrophobic material, and the hydrophobic material can, optionally, be treated with a surfactant or otherwise processed to impart a desired level of wettability and hydrophilicity. For example, the material can be surface treated with about 0.28 weight percent of a surfactant commercially available from the Rohm and Haas Co. under the trade designation Triton X-102. Other suitable surfactants are commercially available from Uniqema Inc., a division of ICI of New Castle, Del., under the trade designation Ahcovel, and from Cognis Corporation of Ambler, Pa., produced in Cincinnati, Ohio, and sold under the trade designation Glucopon 220. The surfactant can be applied by any conventional means, such as spraying, printing, brush coating or the like. The surfactant can be applied to the entire bodyside liner 42 or can be selectively applied to particular sections of the bodyside liner, such as the medial section along the longitudinal centerline.

A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, islands-in-the-sea, or the like. While the outer cover 44 and bodyside liner 42 can include elastomeric materials, it can be desirable in some embodiments for the composite structure to be generally inelastic, where the outer cover, the bodyside liner and the absorbent assembly include materials that are generally not elastomeric.

As noted previously, the illustrated training pant 40 can have front and back side panels 134 and 234 disposed on each side of the absorbent chassis 14 (FIG. 4). These transversely opposed front side panels 134 and transversely opposed back side panels 234 can be permanently bonded to the composite structure 133 of the absorbent chassis 14 and can be releasably attached to one another by a fastening system 80. More particularly, as shown best in FIG. 4, the front side panels 134 can be permanently bonded to and extend transversely beyond the linear side edges 147 of the composite structure 133 along attachment lines 69, and the back side panels 234 can be permanently bonded to and extend transversely beyond the linear side edges of the composite structure along attachment lines 69. The side panels 134 and 234 may be attached using attachment means known to those skilled in the art such as adhesive, thermal or ultrasonic bonding. The side panels 134 and 234 can also be formed as a portion of a component of the composite structure 133, such as the outer cover 44 or the body side liner 42.

Each of the side panels 134 and 234 can include one or more individual, distinct pieces of material. In particular embodiments, for example, each side panel 134 and 234 can include first and second side panel portions that are joined at a seam, with at least one of the portions including an elastomeric material (not shown). Still alternatively, each individual side panel 134 and 234 can include a single piece of material which is folded over upon itself along an intermediate fold line (not shown).

The side panels 134 and 234 desirably include an elastic material capable of stretching in a direction generally parallel to the transverse axis 49 of the training pant 40. In particular embodiments, the front and back side panels 134 and 234 may each include an interior portion 78 disposed between a distal edge 68 and a respective front or back center panel 135 or 235. In the illustrated embodiment in FIG. 4, the interior portions 78 are disposed between the distal edges 68 and the side edges 147 of the rectangular composite structure 133. The elastic material of the side panels 134 and 234 can be disposed in the interior portions 78 to render the side panels elastomeric in a direction generally parallel to the transverse axis 49. Most desirably, each side panel 134 and 234 is elastomeric from a waist end edge 72 to a leg end edge 70. More specifically, individual samples of side panel material, taken between the waist end edge 72 and the leg end edge 70 parallel to the transverse axis 49 and having a length from the attachment line 69 to the distal edge 68 and a width of about 2 centimeters, are all elastomeric.

Suitable elastic materials, as well as one described process of incorporating elastic side panels into a training pant, are described in the following U.S. Pat. Nos. 4,940,464 issued Jul. 10, 1990 to Van Gompel et al.; 5,224,405 issued Jul. 6, 1993 to Pohjola; 5,104,116 issued Apr. 14, 1992 to Pohjola; and 5,046,272 issued Sep. 10, 1991 to Vogt et al.; all of which are incorporated herein by reference. In particular embodiments, the elastic material includes a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8, 1987 in the names of Taylor et al.; all of which are incorporated herein by reference. Alternatively, the side panel material may include other woven or nonwoven materials, such as those described above as being suitable for the outer cover 44 or body side liner 42, or stretchable but inelastic materials.

As described herein, the various components of the training pant 40 can be integrally assembled together employing various types of suitable attachment means, such as adhesive, sonic and thermal bonds or combinations thereof. The resulting product is an absorbent garment 40 including a discreetly thin absorbent material 20 with high absorbent capacity. The pant-like absorbent garment 40 can be sized and tailored for a wide variety of uses including, for example, diapers, training pants, swim wear, incontinence garments, and the like.

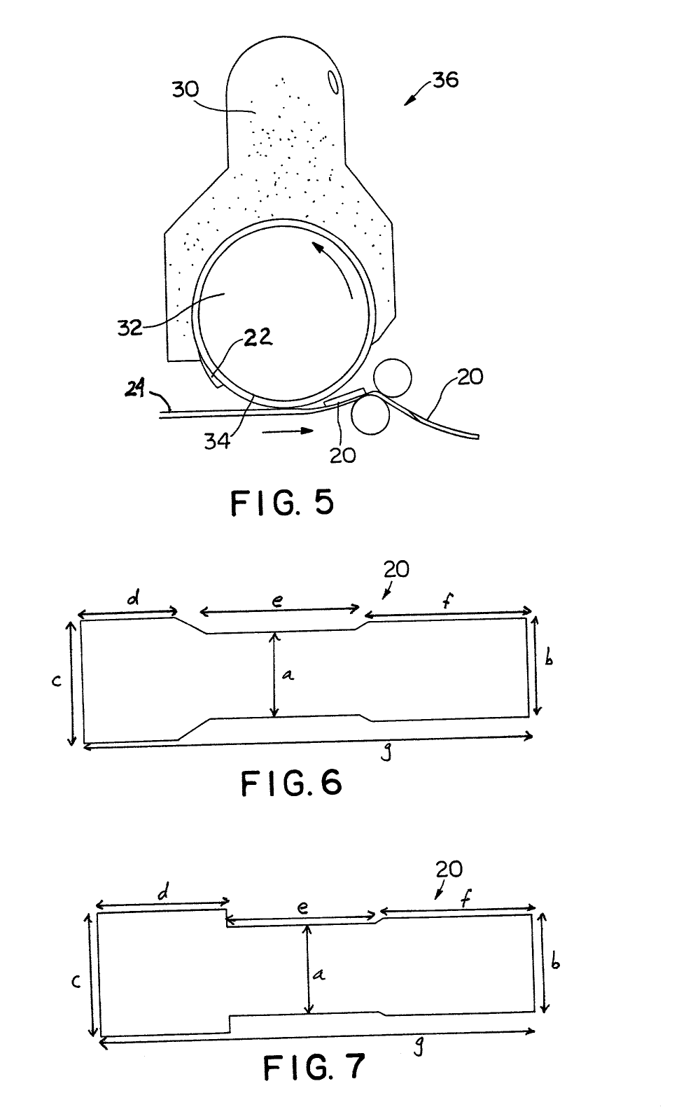

The upper layer 22 of the absorbent material 20 of the invention can be produced using a conventional online absorbent drum former 36, as shown in FIG. 5. More specifically, the SAM and the fluff pulp, and in some cases up to about 4% synthetic fiber, can be mixed in a forming chamber 30 of the drum former 36. A forming screen 34 on a forming drum 32 of the drum former 36 can be either a flat screen or a shaped screen with a differential depth zone.

The lower layer 24 of the absorbent material 20 of the invention can be an offline-formed, homogeneously mixed, air-laid layer, roll good laminate or other offline-formed absorbent composite, examples of which include GELOCK™ laminates available from Gelock International, Dunbridge, Ohio, or NovaThin™ composites from EAM Corporation, Jessup, Ga., or superabsorbent containing airlaid composites from Concert Fabrication of Thurso, Quebec, Canada. Any additional layers 28 or intermediate layers 26 can suitably be either drum-formed like the upper layer 22 or air-laid or other offline-formed composite like the lower layer 24, or produced in any other suitable form.

Online forming of the layered absorbent material 20 can be achieved by unwinding the lower layer 24 in a continuous strip and depositing the upper layer 22 on the lower layer 24, as shown in FIG. 5. Alternatively, the upper layer 22 can be formed and deposited on a carrier device and the lower layer 24 can then be applied to the exposed surface of the upper layer. The composite absorbent material 20 can then be wrapped in tissue and densified by an online de-bulker.

As another alternative, a cut-and-place technique can be used to apply the lower layer material 24 onto the upper layer 22. The lower layer 24 can be applied to a full length of the upper layer 22 or to any portion of the upper layer length. The lower layer 24 can be phased at any position along the length of the upper layer 22. The composite absorbent material 20 can then be wrapped in tissue and densified by an online de-bulker. Alternatively, the lower layer 24 can be applied after the upper layer 22 is wrapped in tissue with or without densification and then laminated to the upper layer using a hot-melt adhesive to form the composite absorbent 20.

During the online forming process of the upper layer 22, the mixture of SAP and pulp fluff can be humidified to improve densification of the resulting upper layer 22 and to possibly provide lower edge compression or stiffness values. The use of heat and humidity in the absorbent composite densification process is taught, for example, in U.S. Pat. No. 6,214,274 issued Apr. 10, 2001, to Melius et al., which is herein incorporated by reference. Furthermore, a pattern can be embossed onto the absorbent material 20 which may also reduce stiffness.

Commercial production rates can be achieved in this process. The densification process can be adjusted to compact the drum-formed SAM/fluff pad to a greater or lesser extent as discussed previously.

The combination of a high density air-laid lower layer 24 and a drum-formed upper layer 22 allows a soft and flexible absorbent material 20 to achieve conformability and comfort while reducing bulk and maintaining intake capability with the full absorbent capacity required of the absorbent product.

### EXAMPLE SET 1

Several combinations of the multi-layer absorbent material of the invention were produced and tested with the Multiple Insult Cradle Test, as described herein. This test assesses the intake and runoff properties of the absorbent structure. The absorbent (fluff) material used was CR 1654, available from U.S. Alliance. The superabsorbent material used was the example pad using FAVOR® SXM 9543 (Code A) or FAVOR® SXM 880 (Code B), available from Stockhausen. Samples were produced from 700 to 1150 grams per square meter (gsm) of absorbent materials with 30 to 60 weight percent of superabsorbent material. A detailed description of an exemplary sample and a control pad appears below in Table 2.

TABLE 2
Sample Compositions

| Sample | Description |
|---|---|
| Code A | A dual layer absorbent pad design. The upper layer was an ultra |
| | thin flat superabsorbent composite of FAVOR ® SXM 9543 that |
| | was drum formed. The lower layer was a NovaThin ™ |
| | absorbent made available by the EAM corporation, Jessup, |
| | Georgia. The lower layer had a density of about 0.38 g/cc. Pad |
| | shape is shown in FIG. 6. Density of the fluff/superabsorbent |
| | layer was about 0.3 g/cc. |
| Code B | Homogeneous (single layer) fluff and FAVOR ® SXM 880 |
| | superabsorbent absorbent pad that was drum formed. Code B is |
| | referenced to be a target control as a measure of performance in |
| | results comparison. Pad shape is shown in FIG. 7. Density of the |
| | pad was about 0.18 g/cc. |

FIGS. 6 and 7 show the respective shapes of absorbent pads of Codes A and B. Exact dimensions for each pad used are as follows. FIG. 6: dimension a=70 mm; b=90 mm; c=115 mm; d=105 mm; e=170 mm; f=160 mm; g=450 mm. FIG. 7: dimension a=70 mm; b=90 mm; c=120 mm; d=115 mm; e=205 mm; f=140 mm; g=470 mm.

Codes A and B were tested for intake/runoff capability using the Multiple Insult Cradle Test. Results are shown below in Table 3.

TABLE 3
Multiple Insult Cradle Test Data

| | Grams of Fluid Runoff Following Insult | | | | | Fluid | Capacity |
|---|---|---|---|---|---|---|---|
| Sample | 100 ml | 75 ml | 75 ml | 75 ml | 75 ml | Held (g) | (g/g) |
| Code A | 23.4 | 1.7 | 5.6 | 16.0 | 24.7 | 337.3 | 8.7 |
| Code B | 2.6 | 2.6 | 13.0 | 17.7 | 26.8 | 345.3 | 8.1 |

Complete products were produced on a commercial pant machine with absorbents as described in Table 2. The absorbent system in each used a 100 gsm surge material of the same composition as described for the absorbents in Table 2. The absorbents of these products were tested n the same manner as those in Table 2; results are shown in Table 4. Code C has the same composition as Coda A in Table 2; Code D has the same composition as Code B in Table 2.

TABLE 4
Multiple Insult Cradle Test Data for Complete Garments

| | Grams of Fluid Runoff Following Insult | | | | | Fluid | Capacity |
|---|---|---|---|---|---|---|---|
| Sample | 100 ml | 75 ml | 75 ml | 75 ml | 75 ml | Held (g) | (g/g) |
| Code C | 0.5 | 0.1 | 3.0 | 11.8 | 23.9 | 374.1 | 331.0 |
| Code D | 0 | 4.4 | 22.4 | 36.4 | 41.6 | 305.9 | 264.3 |

Though in both experiments, the first insult shows higher runoff for the experimental codes (A anc C) than for control codes (B and D), the experimental codes show equivalent or decreased runoff compared to control codes at later insults. These results indicate that absorbent performance of each experimental code exceeds the performance of the corresponding control code in terms of both total amounts of fluid absorbed and overall amounts of fluid runoff.

### EXAMPLE SET 2

In this example, the absorbent composites were converted into test product on a PULL-UPS® Disposable Training Pants machine. The inline forming drum on the machine was used to form the SAM/fluff layer of the composite and additional unwind equipment was placed inline to unwind and add an EAM NovaThin™ composite absorbent pad. All absorbent pads were wrapped in tissue prior to pant assembly. Absorbent material components within these composites included FAVOR® SXM 880 SAM, CR 1654 wood pulp, and NovaThin™ Composite Number 4000255 from EAM Corp., Jessup, Ga. The SAM/fluff component was positioned adjacent the surge material and was densified to about 0.2 g/cc. The EAM composite was adjacent the outer cover, farthest from the user. Pants were made with the following absorbent composites:

Pant A—Control. PULL-UPS ® Disposable Training Pants, size 3, with about 13.0 grams SAM and about 16.5 grams fluff in the absorbent composite.

Pant B—Test pant with about 50% of its absorbent capacity provided by EAM.

Pant C—Test pant with reduced absorbent capacity, and about 65% of that absorbent capacity provided by EAM.

Standard pant materials were used in all products, except for the following:

Surge material for the experimental pants (Pants B and C) was a fiber blend of Bico/PET Bicomponent T-256, T-295 (6/6 denier, 60/40) both from KoSa Corporation, at 80 gsm.

Surge material for the control pant (Pant A) was a through-air-bonded-carded web of a homogenous blend of 60 percent 3 denier bicomponent fiber including a polyester core/polyethylene sheath, and 40 percent 6 denier polyester fiber, both available from KoSa Corporation, at 50 gsm.

Flap elastic was changed from 620 decitex to 740 decitex to match fit parameters of the control.

The pants were tested by 57 male subjects. Percentages of leakage under various conditions were determined for all codes. The median fluid load at failure was determined by the difference between masses of wet and dry products. Descriptions of the resulting test products are shown below in Tables 5 and 6.

TABLE 5
Absorbent Descriptions

| | Absorbent | | | EAM | Saturated | Pant |
|---|---|---|---|---|---|---|
| | Component Weights | | | Size | Capacity | Thickness, |
| Pant | SAM | Fluff | EAM | (mm) | (g) | Front (mm) |
| A | 13.0 | 16.5 | 0.0 | — | 569 | 5.0 |
| B | 7.5 | 9.5 | 14.2 | 76 × 439 | 613 | 4.1 |
| C | 1.5 | 6.5 | 17.4 | 102 × 436 | 523 | 4.2 |

TABLE 6
Subject Testing Results

| | % | % | % | % | Fluid Retained |
|---|---|---|---|---|---|
| | Leakage | Leakage | Leakage | Leakage | Before Leakage |
| Pant | Awake | Nap | Night | Overall | (grams) |
| A | 2.1 | 12.7 | 10.4 | 6.0 | 479 |
| B | 1.4 | 4.7 | 11.1 | 5.0 | 513 |
| C | 3.0 | 10.3 | 14.2 | 7.6 | 455 |

The data in Table 6 indicate that Pant B exhibited leakage control equivalent to Pant A (control), in spite of a reduction in front absorbent thickness of 18%. Pant C, with its reduced absorbent capacity, had more leakage than control Pant A, but its performance was not found to be statistically different.

### Liquid Saturated Retention Capacity Test Procedure

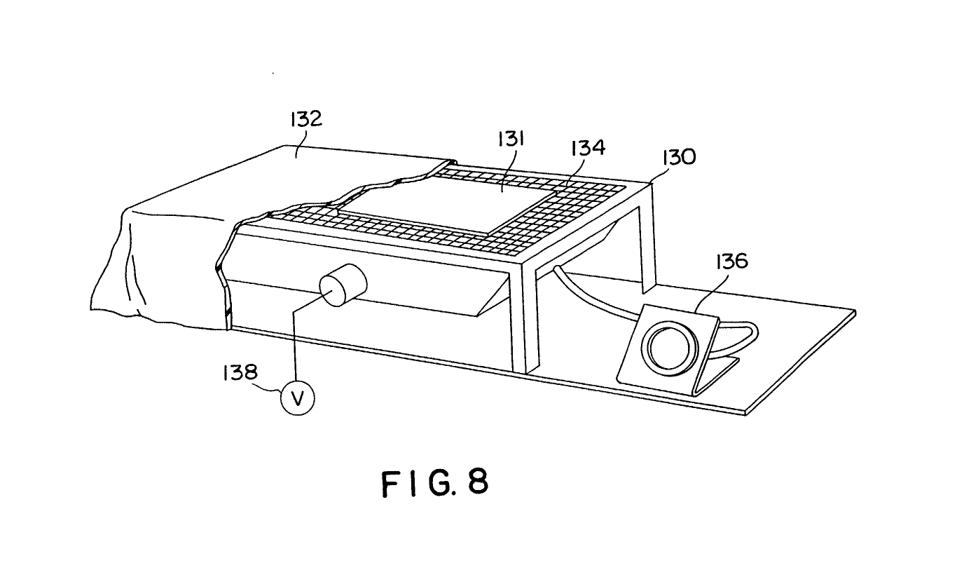

The liquid saturated retention capacity is determined as follows. The material to be tested, having a moisture content of less than about 7 weight percent, is weighed and submerged in an excess quantity of a 0.9 weight percent aqueous saline solution at room temperature (about 23 degrees Celsius). The material to be tested is allowed to remain submerged for about 20 minutes. After the 20 minute submerging, the material is removed and, referring to FIG. 8, placed on a TEFLON™ coated fiberglass screen 134 having 0.25 inch (0.6 cm) openings (commercially available from Taconic Plastics Inc., Petersburg, N.Y.) which, in turn, is placed on a vacuum box 130 and covered with a flexible rubber dam material 132. A vacuum of about 0.5 pound per square inch (about 3.5 kilopascals) is drawn on the vacuum box for a period of about 5 minutes with the use of , for example, a vacuum gauge 136 and a vacuum pump 138. The material 131 being tested is then removed from the screen and weighed. The amount of liquid retained by the material being tested is determined by subtracting the dry weight of the material from the wet weight of the material (after application of the vacuum), and is reported as the absolute liquid saturated retention capacity in grams of liquid retained. If desired, the weight of liquid retained may be converted to liquid volume by using the density of the test liquid, and is reported as the liquid saturated retention capacity in milliliters of liquid retained. For relative comparisons, this absolute liquid saturated retention capacity value can be divided by the dry weight of the material 131 to give the specific liquid saturated retention capacity in grams of liquid retained per gram of tested material. If material, such as hydrogel-forming polymeric material or fiber, is drawn through the fiberglass screen while on the vacuum box, a screen having smaller openings should be used. Alternatively, a piece of tea bag or similar material can be placed between the material and the screen and the final value adjusted for the liquid retained by the tea bag or similar material.

### Absorbency Under Load (AUL) Test Procedure

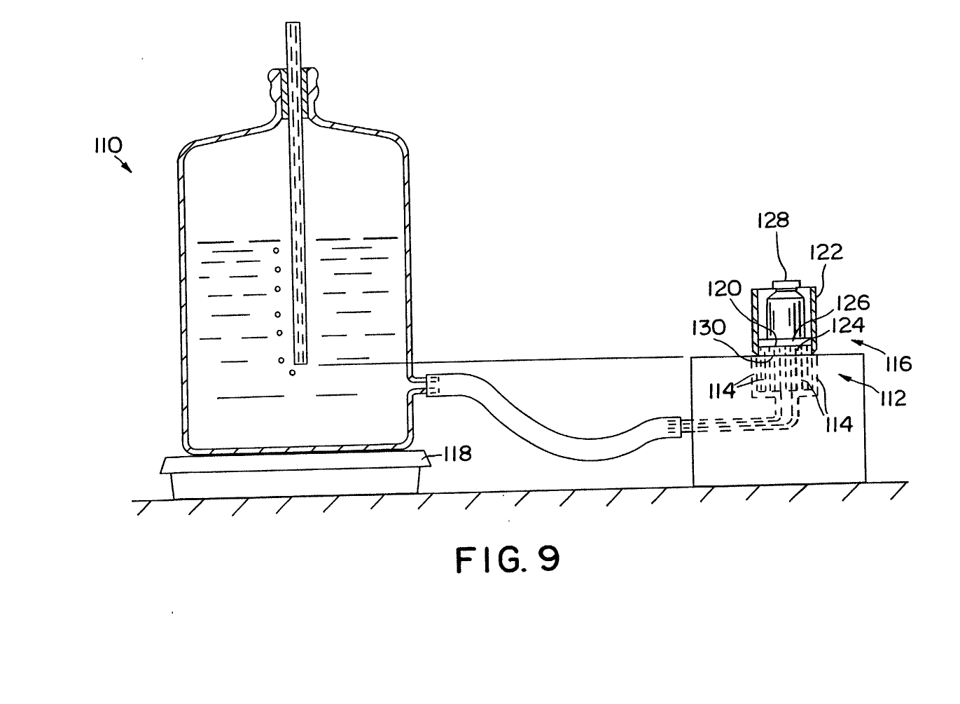

The ability of a superabsorbent material to absorb a liquid while under a load is determined as follows. With reference to FIG. 9, a Demand Absorbency Tester (DAT) 110 is used, which is similar to the GATS (Gravimetric Absorbency Test System), available from M/K systems, Danners, Mass., as well as the system described by Lichstein at pages 129-142 of the INDA Technological Symposium Proceedings, Mar. 1974. A porous plate 112 is used, having ports 114 confined within a 2.5 centimeter diameter area and covered by the Absorbency Under Load (AUL) apparatus 116. An electrobalance 118 is used to measure the flow of fluid into the superabsorbent particles 120. For this test, the fluid employed is an aqueous solution containing 0.9 weight percent sodium chloride used at room temperature (approximately 23° Celsius).

The special AUL apparatus 116 used to contain the superabsorbent particles comprises a cylinder 122 made from 1 inch (2.54 centimeters) inside diameter thermoplastic tubing which is machined-out slightly to be sure of concentricity. A 100 mesh stainless steel wire cloth 124 is adhered on the bottom of cylinder 122 by means of an adhesive. Alternatively, the stainless steel wire cloth 124 can be fused to the bottom of cylinder 122 by heating the wire cloth in a flame until red hot, after which the cylinder is held onto the cloth until cooled. A soldering iron can be used to touch up the seal if unsuccessful or if it breaks. Care must be taken to maintain a flat, smooth bottom, and not distort the inside of the cylinder. A 4.4 gram piston 126 is made from 1 inch diameter solid material (e.g. Plexiglass™) and is machined to closely fit without binding in the cylinder 122. The piston 126 is used to provide the restraining load of 0.01 pound per square inch. A weight 128 is used to provide the greater degrees of restraining load. As discussed above, the greater restraining loads are 0.29 pound per square inch, 0.57 pound per square inch, and 0.90 pound per square inch. Accordingly, a 100, 200, and 317 gram weight is used to provide the respective restraining loads (in addition to the 4.4 gram piston 126). A sample of superabsorbent particles weighing 0.160 (±005) gram is utilized for testing AUL. The sample is taken from granules which are pre-screened through U.S. standard 30 mesh and retained on U.S. standard 50 mesh (300-600 microns). The particles, when tested, have a moisture content of less than about 5 weight percent.

This test is initiated by placing a 3 centimeter diameter GF/A glass filter paper 130 onto the plate 112. The paper is sized to be larger than the internal diameter and smaller than the outside diameter of the cylinder 122 to ensure good contact while eliminating evaporation over the ports 114 of the DAT 110 and then allowing saturation to occur. The particles 120 are weighed on weighing paper and placed on the wire cloth 124 at the bottom of the AUL apparatus 116. The apparatus 116 is shaken to level the particles 120 on the wire cloth 124. Care is taken to be sure no particles are clinging to the wall of the cylinder 122. After carefully placing, without pressing, the piston 126 and, optionally, weight 128 on the particles 120 in the cylinder 122, the AUL apparatus 116 is placed on the glass filter paper 130. The amount (in grams) of fluid picked up is monitored as a function of time either directly by hand, with a strip-chart recorder, or directly into a data acquisition or personal computer system.

The amount (in grams) of fluid picked up after 60 minutes, divided by the dry weight of the sample (0.160 gram) is the AUL value in grams of fluid picked up per gram of sample (g/g). The rate of fluid picked up can also be measured. Two checks can be made to ensure the accuracy of the instantaneous final readout. First, the height the piston 126 rises, multiplied by the cross-sectional area of the cylinder 122 should equal the volume of fluid picked up. Second, the AUL apparatus 116 can be weighed before and after the test, and the difference in weight should nearly equal the weight of fluid picked up. A minimum of three tests are performed on a given sample and averaged to assign an AUL value.

### Bulk and Density Test Procedures

A region of the absorbent pad to be tested is placed under a 0.2 psi weight, and the bulk of the absorbent in this region is recorded. The area under compression should be larger than a 2-inch by 2-inch (5.08 cm by 5.08 cm) square. A suitable tester for absorbent bulk is a Starret-type bulk tester equipped with a 3-inch diameter brass foot that applies a weight of 0.2 psi. The area under compression is marked around the perimeter of the weight while the weight is in place. The weight is removed, and a 2-inch by 2-inch square is cut out from within the outlined region, such as by a die cut. Any tissue present on the absorbent pad is removed, and the square is weighed. The density is determined by the following calculation: density mass of absorbent in g/(5.08 cm)² x (bulk in cm).

### Centrifuge Retention Capacity Test Procedure (CRC Test)

As used herein, the Centrifugal Retention Capacity (CRC) is a measure of the absorbent capacity of the superabsorbent material after being subjected to centrifugation under controlled conditions. The superabsorbent sample to be tested is taken from superabsorbent material which is prescreened through U.S. standard #30 mesh and retained on U.S. standard #50 mesh. The superabsorbent material therefore has a particle size of between 300 and 600 microns. The particles can be prescreened by hand or automatically.

The CRC can be measured by placing 0.200 grams of the sample material to be tested (moisture content of less than 5 weight percent) into a water-permeable bag which will contain the sample while allowing the test solution (0.9 percent NaCl solution) to be freely absorbed by the sample. A heat-sealable tea bag material (grade 542, commercially available from Kimberly-Clark Corporation, Neenah, Wis.) works well for most applications. The bag is formed by folding a 5-inch by 3-inch sample of the bag material in half and heat-sealing two of the open edges to form a 2.5-inch by 3-inch rectangular pouch. The heat seals should be about 0.25 inch inside the edge of the material. After the sample is placed in the pouch, the remaining open edge of the pouch is also heat-sealed. Empty bags are also made to be tested with the sample bags as controls. Three sample bags are tested for each superabsorbent material.

The sealed bags are placed between two TEFLON® coated fiberglass screens having ¼ inch openings (Taconic Plastics, Inc., Petersburg, N.Y.) and submerged in a pan of 0.9 percent NaCl solution at 73.4° ±2° Fahrenheit, making sure that the screens are held down until the bags are completely wetted. After wetting, the samples remain in the solution for 30 minutes, at which time they are removed from the solution and temporarily laid on a nonabsorbent flat surface.

The wet bags are then placed into the basket of a suitable centrifuge capable of subjecting the samples to a g-force of 350. (A suitable centrifuge is a Clay Adams Dynac II, model #0103, having a water collection basket, digital rpm gauge, and machined drainage basket adapted to hold and drain the flat bag samples). The samples must be placed in opposing positions within the centrifuge to balance the basket when spinning. The bags are centrifuged at a target of 1600 rpm, but within the range of 1500-1900 rpm, for 3 minutes (target g-force of 350). The bags are removed and weighed, with the empty bags (controls) being weighed first, followed by the bags containing superabsorbent material. The amount of fluid absorbed and retained by the superabsorbent material, taking into account the fluid retained by the bag material alone, is the Centrifugal Retention Capacity of the superabsorbent material, expressed as grams of fluid per gram of superabsorbent material.

### Superabsorbent Gel Bed Permeability Test

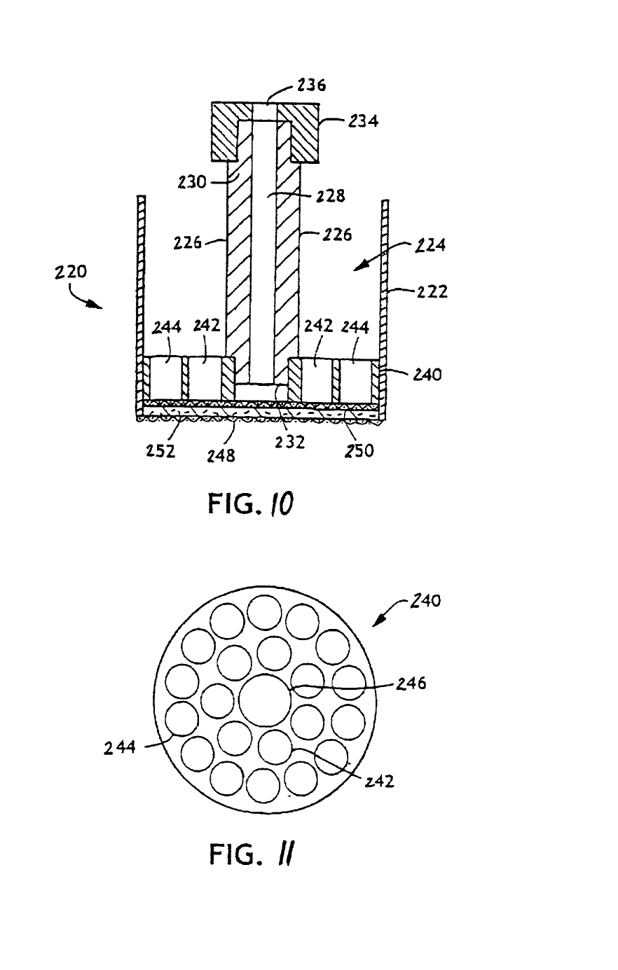

A suitable piston/cylinder apparatus for performing the Gel Bed Permeability (GBP) test is shown in FIGS. 10 and 11. Referring to FIG. 10, an apparatus 220 consists of a cylinder 222 and a piston (generally indicated as 224). As shown in FIG. 10, the piston 224 consists of a cylindrical LEXAN shaft 226 having a concentric cylindrical hole 228 bored down the longitudinal axis of the shaft. Both ends of the shaft 226 are machined to provide first and second ends 230, 232. A weight 234 rests on the first end 230 and has a cylindrical hole 236 bored through the center thereof. Inserted on the second end 232 is a circular piston head 240. The piston head 240 is sized so as to vertically move inside the cylinder 222.

As shown in FIG. 11, the piston head 240 is provided with inner and outer concentric rings containing seven and fourteen approximately 0.375 inch (0.95 cm) cylindrical holes, respectively (indicated generally by arrows 242 and 244 ). The holes in each of these concentric rings are bored from the top to bottom of the piston head 240. The piston head 240 also has a cylindrical hole 246 bored in the center thereof to receive the second end 232 of the shaft 226.

Attached to the bottom end of the cylinder 222 is a No. 400 mesh stainless steel cloth screen 248 that is biaxially stretched to a tautness prior to attachment. Attached to the bottom end of the piston head 240 is a No. 400 mesh stainless steel cloth screen 250 that is biaxially stretched to a tautness prior to attachment. A sample of adsorbent material 252 is supported on the screen 248.

The cylinder 222 is bored from a transparent LEXAN rod or equivalent and has an inner diameter of 6.00 cm (area=28.27 cm²), a wall thickness of approximately 0.5 cm, and a height of approximately 5.0 cm. The piston head 240 is machined from a LEXAN rod. It has a height of approximately 0.625 inches (1.59 cm) and a diameter sized such that it fits within the cylinder 222 with minimum wall clearances, but still slides freely. A hole 246 in the center of the piston head 240 has a threaded 0.625 inch (1.59 cm) opening (18 threads/inch) for the second end 232 of the shaft 226.

The shaft 226 is machined from a LEXAN rod and has an outer diameter of 0.875 inches (2.22 cm) and an inner diameter of 0.250 inches (0.64 cm). The second end 232 is approximately 0.5 inches (1.27 cm) long and is threaded to match the hole 246 in the piston head 240. The first end 230 is approximately 1 inch (2.54 cm) long and 0.623 inches (1.58 cm) in diameter, forming an annular shoulder to support the stainless steel weight 234.

The annular stainless steel weight 234 has an inner diameter of 0.625 inches (1.59 cm), so that it slips onto the first end 230 of the shaft 226 and rests on the annular shoulder formed therein. The combined weight of the piston 224 and the weight 134 equals approximately 596 g, which corresponds to a pressure of 0.30 psi (20,685 dynes/cm), for an area of 28.27 cm². When fluids flow through the piston/cylinder apparatus, the cylinder 222 generally rests on a 16-mesh, rigid stainless-steel support screen (not shown) or equivalent.

The piston and weight are placed in an empty cylinder to obtain a measurement from the bottom of the weight to the top of the cylinder. This measurement is taken using a caliper readable to 0.01 mm. This measurement will later be used to calculate the height of the bed of the sample of adsorbent material 252. It is important to measure each cylinder empty and keep track of which piston and weight were used. The same piston and weight should be used for measurement when the sample of adsorbent material is swollen.

The adsorbent layer used for GBP measurements is formed by swelling approximately 0.9 g of a sample of adsorbent material in the GBP cylinder apparatus (dry adsorbent material should be spread evenly over the screen of the cylinder prior to swelling) with a fluid, typically 0.9% (w/v) aqueous NaCl, for a time period of approximately 15 minutes. The sample of adsorbent material is taken from a population of adsorbent material that is prescreened through U.S. standard 30 mesh and retained on U.S. standard 50 mesh. The adsorbent material, therefore, has a particle size of between 300 and 600 microns. The particles may be prescreened by hand or automatically prescreened with, for example, a Ro-Tap Mechanical Sieve Shaker Model B, commercially available from W. S. Tyler, Inc., Mentor, Ohio, USA.

At the end of the 15 minute period, the cylinder is removed from the fluid and the piston/weight assembly is placed on the sample of adsorbent material. The thickness of the swollen sample of adsorbent material is determined by measuring from the bottom of the weight to top of the cylinder with a micrometer. The value obtained when taking this measurement with the empty cylinder is subtracted from the value obtained after swelling the sample of adsorbent material. The resulting value is the height of the bed of the swollen sample of adsorbent material, H.

The GBP measurement is initiated by adding the fluid to the cylinder 222 until the fluid attains a height of 4.0 cm above the bottom of the sample of adsorbent material 252. This fluid height is maintained throughout the test. The quantity of fluid passing through the sample of adsorbent material 252 versus time is measured gravimetrically. Data points are collected every second for the first two minutes of the test and every two seconds for the remainder. When the data are plotted as quantity of fluid passing through the bed of the sample of adsorbent material versus time, it becomes clear to one skilled in the art when a steady flow rate has been attained.

Only data collected once the flow rate has become steady is used in the flow rate calculation. The flow rate, Q, through the sample of adsorbent material 252, is determined in units of g/s by a linear least-square fit of fluid passing through the sample of adsorbent material (in grams) versus time (in seconds). Permeability in cm² is obtained by the following equation: K=[Q*(H*μ)]/[A*p*P], where K=Gel Bed Permeability (cm²); Q=flow rate (g/sec); H=height of bed of sample of adsorbent material (cm); μ=liquid viscosity (poise); A=cross-sectional area for liquid flow (cm²); p=liquid density (g/cm3); and P=hydrostatic pressure (dynes/cm²) (normally approximately 3,923 dynes/cm²).

### Multiple Insult Cradle Test Procedure

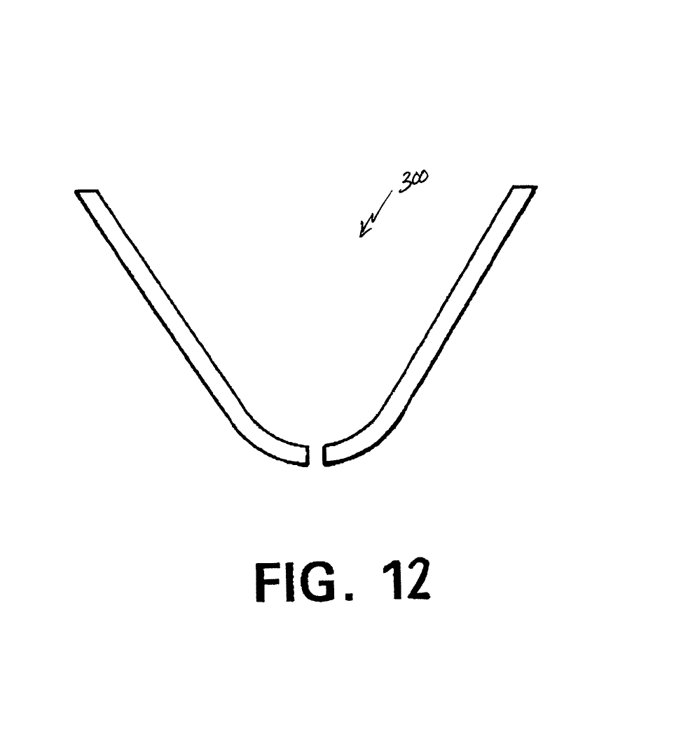

In this test, an absorbent material is placed in an acrylic cradle to simulate body curvature of a wearer. Such a cradle 300 is illustrated in FIG. 12. The cradle 300 has a width into the page of the drawing, as shown, of 33 cm and the ends are blocked off. The cradle 300 has a height of 19 cm, an inner distance between the upper arms of 30.5 cm, and an angle between the upper arms of 60 degrees. The cradle 300 has a 6.5 mm wide slot at the lowest point, which runs the length of the cradle into the page.

The test is designed to determine how quickly an absorbent material is able to absorb an insult as opposed to allowing the insulting fluid to run off the sample without being absorbed. The cradle 300 is set at a slight incline (described below) to minimize pooling of fluid. The material to be tested is weighed. An average weight of any tissue wrap should be determined by individually weighing at least ten pieces of tissue of the same basis weight and dimensions as used to wrap the material, and averaging the individual tissue weights. The average tissue weight is subtracted from the overall weight of the dry material. The thicknesses of the material at the front end, middle and back end are determined using the method described elsewhere under Bulk and Density Tests.

A piece of surge material is cut to a dimension of 240 mm by 62 mm and placed on top of each piece of absorbent material so that the surge is centered and the front edge is located 47 mm from the front end of the absorbent, on the top surface of the top layer. In these tests, a surge layer with a basis weight of 85 gsm, composed of 60 wt % 2.0 denier T-256 type bicomponent fiber and 40 wt % of 3.0 denier polyester fiber from KoSa, was used for all samples. The surge is attached to the top surface of the top layer using double-sided tape along its long edges. The insult point is marked at 5.5 inches from the front of the absorbent composite, and the center of the absorbent composite is marked.

Materials are placed in the cradle 300, with the center of the absorbent composite at the bottom slot of the cradle. A plastic capture container of known weight is placed under the cradle 300. One leg of the cradle 300 is propped up so that the transverse axis of the absorbent, as illustrated in FIG. 4, is held at an angle of 7.5° above the horizontal.

A timer set to 20 minutes is started, and the material is immediately insulted with 100 mL of 0.9 w/v % saline solution at a rate of about 10 mL/sec. The nozzle of the saline delivery pump is held about 0.5 cm away from the surface of the absorbent composite and with the orifice normal to the absorbent composite surface, at the marked insult point.

Following delivery of the insult, the mass of any runoff fluid in the capture container is determined and recorded. Any runoff that does not land in the capture container is wiped up with a weighed paper towel, after which the wet towel is again weighed. The mass difference between the towel in wet and dry states is added to the runoff mass in the container. After the timed 20 minutes elapses, the insult and runoff procedure is repeated, using 75 mL of saline solution. Additional 75 mL insults are given at 20 minute intervals and the runoff is weighed, until a total of five insults have been delivered. Following the fifth insult, the wet weight of the sample is determined.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An absorbent material, comprising:
an upper layer including pulp fluff and superabsorbent material;
a lower layer including pulp fluff and superabsorbent material;
wherein the absorbent material has a thickness in a range of between 0.5 and 7.5 millimeters, and an absorbent capacity between about 14 and 40 grams 0.9 w/v % saline solution per gram of absorbent material, and the lower layer has a greater density than the upper layer.

2. The absorbent material of claim 1, wherein the upper layer has a density in a range of between 0.05 and 0.45 grams per cubic centimeter.

3. The absorbent material of claim 1, wherein the upper layer has a density in a range of between 0.1I and 0.4 grams per cubic centimeter.

4. The absorbent material of claim 1, wherein the lower layer has a density in a range of between 0.15 and 0.55 grams per cubic centimeter.

5. The absorbent material of claim 1, wherein the lower layer has a density in a range of between 0.2 and 0.5 grams per cubic centimeter.

6. The absorbent material of claim 1, wherein a difference between the density of the upper layer and the density of the lower layer is in a range of between 0.01 and 0.40 grams per cubic centimeter.

7. The absorbent material of claim 1, wherein a difference between the density of the upper layer and the density of the lower layer is in a range of between 0.05 and 0.35 grams per cubic centimeter.

8. The absorbent material of claim 1, wherein a difference between the density of the upper layer and the density of the lower layer is in a range of between 0.15 and 0.25 grams per cubic centimeter.

9. The absorbent material of claim 1, wherein the upper layer comprises between 20 and 70 wt % superabsorbent material.

10. The absorbent material of claim 1, wherein the lower layer comprises between 10 and 80 wt % superabsorbent material.

11. The absorbent material of claim 1, wherein the absorbent material has a thickness in a range of 1 to 7 millimeters.

12. The absorbent material of claim 1, wherein the absorbent material has an absorbent capacity of at least 16 grams 0.9 w/v % saline solution per gram of absorbent material.

13. The absorbent material of claim 1, wherein the absorbent material has an absorbent capacity of at least 18 grams 0.9 w/v % saline solution per gram of absorbent material.

14. The absorbent material of claim 1, further comprising an intermediate layer between the upper layer and the lower layer, wherein the intermediate layer includes pulp fluff and superabsorbent material.

15. The absorbent material of claim 1, further comprising an additional layer on top of the upper layer, wherein the additional layer includes pulp fluff and superabsorbent material.

16. The absorbent material of claim 1, where in the upper layer comprises a bottom surface facing the lower layer, the lower layer comprises a top surface facing the bottom surface of the upper layer, and a surface area of the bottom surface of the upper layer is greater than a surface area of the top surface of the lower layer.

17. The absorbent material of claim 1, wherein the lower layer is discontinuous.

18. The absorbent material of claim 1, wherein the upper layer is drum-formed.

19. The absorbent material of claim 1, wherein the lower layer is air-laid.

20. An absorbent article comprising the absorbent material of claim 1.

21. A diaper comprising the absorbent material of claim 1.

22. A training pant comprising the absorbent material of claim 1.

23. A feminine hygiene product comprising the absorbent material of claim 1.

24. An incontinence product comprising the absorbent material of claim 1.

25. A swim wear garment comprising the absorbent material of claim 1.

26. An absorbent material, comprising:
an upper layer including pulp fluff and superabsorbent material;
a lower layer including pulp fluff and superabsorbent material;
wherein the absorbent material has a thickness in a range of between 0.5 and 7.5 millimeters, and an absorbent capacity between about 14 and 40 grams 0.9 w/v % saline solution per gram of absorbent material, and the lower layer has a density equal to a density of the upper layer.

27. An absorbent garment, comprising:
a chassis defining a waist opening and first and second leg openings;
the chassis including at least a liquid-permeable body side liner, an absorbent assembly and a substantially liquid-impermeable outer cover layer;
wherein the absorbent assembly includes an upper layer of pulp fluff combined with superabsorbent material, a lower layer of pulp fluff combined with superabsorbent material, the lower layer has a greater density than the upper layer, and the absorbent assembly has a thickness in a range of between 0.5 and 7.5 millimeters and an absorbent capacity between about 14 and 40 grams 0.9 w/v % saline solution per gram of absorbent material.

28. The absorbent garment of claim 27, wherein the upper layer of the absorbent assembly has a density in a range of between 0.1 and 0.4 grams per cubic centimeter.

29. The absorbent garment of claim 27, wherein the lower layer of the absorbent assembly has a density in a range of between 0.2 and 0.5 grams per cubic centimeter.

30. The absorbent garment of claim 27, wherein the upper layer of the absorbent assembly comprises between 20 and 70 wt % superabsorbent material.

31. The absorbent garment of claim 27, wherein the lower layer of the absorbent assembly comprises between 10 and 80 wt % superabsorbent material.

32. The absorbent garment of claim 27, wherein the absorbent assembly has a thickness in a range of 1 to 7 millimeters.

33. The absorbent garment of claim 27, wherein the absorbent material has an absorbent capacity of at least 16 grams 0.9 w/v % saline solution per gram of absorbent material.

34. The absorbent material of claim 27, wherein the absorbent material has an absorbent capacity of at least 18 grams 0.9 w/v % saline solution per gram of absorbent material.

35. The absorbent garment of claim 27, wherein the absorbent assembly further comprises an intermediate layer between the upper layer and the lower layer, wherein the intermediate layer includes pulp fluff and superabsorbent material.

36. The absorbent garment of claim 35, wherein the intermediate layer is drum-formed.

37. The absorbent garment of claim 35, wherein the intermediate layer is air-laid.

38. The absorbent garment of claim 27, wherein the absorbent assembly further comprises an additional layer on top of the upper layer, wherein the additional layer includes pulp fluff and superabsorbent material.

39. The absorbent garment of claim 38, wherein the additional layer is drum-formed.

40. The absorbent garment of claim 27, wherein the upper layer of the absorbent assembly is drum-formed.

41. The absorbent garment of claim 27, wherein the lower layer of the absorbent assembly is air-laid.

42. The absorbent garment of claim 27, wherein the lower layer is discontinuous and is placed in desired locations of the absorbent assembly.

43. An absorbent garment, comprising:
a chassis defining a waist opening and first and second leg openings;
the chassis including at least a liquid-permeable body side liner, an absorbent assembly and a substantially liquid-impermeable outer cover layer;
wherein the absorbent assembly includes an upper layer of pulp fluff combined with superabsorbent material, a lower layer of pulp fluff combined with superabsorbent material, the lower layer has a density equal to a density of the upper layer, and the absorbent assembly has a thickness in a range of between 0.5 and 7.5 millimeters and an absorbent capacity between about 14 and 40 grams 0.9 w/v % saline solution per gram of absorbent material.

44. A method of making a composite absorbent material, comprising the steps of:
providing a first absorbent material;
homogeneously mixing superabsorbent material and fluff pulp in a forming chamber of an online drum former;
forming a second absorbent material from the homogeneously mixed superabsorbent material and fluff pulp as the homogeneously mixed superabsorbent material and fluff pulp exits the forming chamber onto a forming screen on a forming drum of the drum former;
compacting the second absorbent material to a density of at least 0.25 grams per cubic centimeter after the second absorbent material leaves the forming screen; and
bonding the first absorbent material and the second absorbent material together.

45. The method of claim 44, wherein the first absorbent material is air-laid.

46. The method of claim 44, wherein the first absorbent material comprises a roll good laminate.

47. The method of claim 44, further comprising the step of depositing the second absorbent material onto the first absorbent material prior to bonding the first absorbent material and the second absorbent material together.

48. The method of claim 44, further comprising the step of depositing the second absorbent material onto a carrier device and applying the first absorbent material to the second absorbent material on the carrier device prior to bonding the first absorbent material and the second absorbent material together.

49. The method of claim 44, further comprising the step of wrapping the composite absorbent material in tissue.

50. The method of claim 44, further comprising the step of wrapping the second absorbent material in tissue.

51. The method of claim 44, wherein the first absorbent material is applied discontinuously to portions of the second absorbent material prior to bonding the first absorbent material and the second absorbent material together.

52. The method of claim 44, further comprising the step of directing an additional mass of the homogeneously mixed superabsorbent material and pulp fluff into at least one area of the first absorbent material.

53. The method of claim 44, further comprising the step of embossing a pattern onto the composite absorbent material.

54. The method of claim 44, further comprising the step of compacting the composite absorbent material to a thickness of between 0.5 and 3.0 millimeters.

55. The method of claim 44, further comprising the step of bonding a third absorbent material to the composite absorbent material.

56. The method of claim 44, further comprising the step of bonding a drum-formed third absorbent material to the composite absorbent material.
